(19) United States
(12) Patent Application Publication
(10) Pub. No.: US 2023/0218227 A1
(43) Pub. Date: Jul. 13, 2023

(54) SENSOR ARRANGEMENT FOR ATTACHMENT TO A HEAD OF A USER

(71) Applicant: Robert Bosch GmbH, Stuttgart (DE)

(72) Inventors: Johannes Meyer, Magstadt (DE); Thomas Alexander Schlebusch, Renningen (DE)

(21) Appl. No.: 18/151,594

(22) Filed: Jan. 9, 2023

(30) Foreign Application Priority Data
Jan. 13, 2022 (DE) 10 2022 200 312.8

(51) Int. Cl. A61B 5/00

(52) U.S. Cl. CPC A61B 5/6803

(57) ABSTRACT

A sensor arrangement for attachment to a head of a user. The sensor arrangement includes at least one first electrical sensor and/or at least one second optical sensor. The sensor arrangement further comprises a support structure designed to support the first sensor and/or second sensor on the head of the user such that the first sensor and/or second sensor is arranged in an area of the ear. The sensor arrangement further includes at least one connecting element made of an elastic material for the purpose of at least indirect connection between the first sensor and/or second sensor and the scalp of the user.

### CROSS REFERENCE

The present application claims the benefit under 35 U.S.C. § 119 of German Patent Application No. DE 10 2022 200 312.8 filed on Jan. 13, 2022, which is expressly incorporated herein by reference in its entirety.

### FIELD

The present invention relates to a sensor arrangement for attachment to a head of a user, to eyeglasses having the sensor arrangement, and to a method used for manufacturing a sensor arrangement for attachment to a head of a user.

### BACKGROUND INFORMATION

German Patent Application No. DE 10 2015 205 921 A1 describes data eyeglasses, on the temples of which capacitive sensors are arranged in the ear region such that skin contact is made.

An object of the present invention is to develop a sensor arrangement that is optimized to the head movements of the user.

### SUMMARY

According to the present invention, a sensor arrangement for attachment to a head of a user is provided. According to an example embodiment of the present invention, the sensor arrangement for attachment to a head of a user comprises at least one first electrical sensor and/or at least one second optical sensor. In addition, the sensor arrangement comprises a support structure designed to support and/or attach the first and/or second sensor to the head of the user such that the first and/or second sensor is arranged on an area of the ear, in particular in an area between one ear and the head. Preferably, the support structure is at least one headphone or one temple of the eyeglasses. The sensor arrangement comprises at least one connecting element made of an elastic material for the purpose of at least an indirect connection, in particular an attachment, between the first sensor and/or second sensor and the scalp of the user. By using the connecting element that is made of the elastic material, the support structure, and thus also the first sensor and/or second sensor, is attached more securely to the head of the user. The risk of the sensor arrangement slipping (thus resulting in erroneous sensor data) during user movement is hereby reduced.

Preferably, the support structure is designed as a first temple of the eyeglasses, in particular data eyeglasses, of the user. The first sensor and/or second sensor is in this case integrated into the first temple and/or arranged on an outer side of the first temple. The first temple typically features a stable fit on the head of the user and extends to the area behind their ear. The temple is usually also in contact with the skin in this location. The temple thus meets all of the criteria for a support unit of the sensor arrangement.

Preferably, according to an example embodiment of the present invention, the first sensor is designed as an accelerometer and/or a rotation rate sensor. The first sensor benefits from a particularly fixed connection with the head by means of the connecting element, so that signals resulting from movement of the frame of the eyeglasses are minimized. The accelerometer and/or rotation rate sensor is preferably used to detect head and body movements, in particular gestures such as nodding and/or back-and-forth movements of the head. These types of sensors can further serve as pedometers. By arranging the first sensor in the region between the ear and the head, the jaw muscle movements of the user are easily detectable. In this context, the first sensor can serve to differentiate the activity of the user, e.g., between “eating” and “speaking”. Alternatively or additionally, the first sensor is designed as an EMG sensor. Further alternatively or additionally, the first sensor is designed as an ECG sensor. Since the moisture in the region of the connecting element is not dissipated, the electrical connection benefits from a slight moisture buildup on the skin, so that the skin impedance is reduced and the electrical connection is further improved. Further alternatively or additionally, the first sensor is designed as a microphone. Via coupling of the first sensor with the structure-borne sound of the jawbone, the sensor can be used for the voice control of a pair of data eyeglasses.

Preferably, according to an example embodiment of the present invention, the second sensor is designed as a PPG (photoplethysmography) sensor. Alternatively or additionally, the second sensor is designed as a functional near-infrared (fNIR) sensor.

Preferably, the sensor arrangement comprises at least a third electrical sensor. In this context, the support structure is designed to support the third sensor on the head of the user such that the third sensor is arranged at a distance from the head, in particular the scalp, of the user. The third sensor is designed as a microphone and/or as an accelerometer. Preferably, the third sensor is not arranged in the area between the ear and the scalp. The electrical sensor, which is arranged separately and at a distance from the scalp, provides the option of using said sensor to record ambient noises or measure ambient air pressures. This separately acquired sensor data can be processed separately or processed together with the sensor data from the first sensor and/or second sensor. For example, signal fusion or channel separation can be performed so that environmental factors and head factors are separated. For example, a major axis transformation of the signals can in this case be performed. The result is improved functioning of the downstream function blocks, e.g., activity differentiation or voice recognition.

Preferably, according to an example embodiment of the present invention, the connecting element is designed as a gel pad. Such a gel pad features the elasticity necessary to retain the support structure in a stable position during movement by the user. Preferably, the gel pad is manufactured as a separate component and is connected to the support structure by means of an adhesive. Alternatively, the gel pad is preferably integrated into the support structure, which ensures particularly effective sensor coupling. The latter can be achieved by, e.g., overmolding the temple that is acting as the support structure, as well as the sensors integrated into it. Alternatively, the gel pad is preferably designed as a liquid-filled membrane. In this context, the gel pad is in particular designed as a silicon oil-filled bag having a membrane for connection with the head. Preferably, the first sensor is arranged on an outer side of the support structure and is at least partially enclosed by the gel pad. In this context, the outer side of the support structure is in particular understood to mean the surface of the support structure that is arranged facing the scalp of the user.

Preferably, the second sensor is introduced into a cavity of the support structure. The connecting element thereby closes the cavity in an outward direction. Preferably, an additional waveguide is provided for guiding the light waves between the connecting element and the second sensor. Alternatively, the second sensor is integrated into the external surface of the support structure.

Preferably, according to an example embodiment of the present invention, the connecting element is integrated into the support structure, in particular an outer side of the support structure. This may be achieved by, e.g., overmolding the support structure as well as the sensors integrated into it.

Preferably, according to an example embodiment of the present invention, the connecting element is at least partially made of an electrically conductive material. In this context, the connecting element is preferably at least partially made of carbon powder, carbon nanoparticles, and/or metal particles. Alternatively or additionally, the connecting element is at least partially made of a carbon-admixed two-component polysiloxane. This material is particularly cast by means of injection molding and then thermally crosslinked.

Preferably, according to an example embodiment of the present invention, the connecting element is at least partially made of a transparent, in particular translucent, material. In this context, the connecting element is in particular designed as a silicone oil-filled, translucent pouch, in particular a membrane. Preferably, the connecting element is at least partially made of a material which is transparent or non-transparent, depending on the wavelength of the light.

Preferably, according to an example embodiment of the present invention, the connecting element comprises a first electrical region made of the electrically conductive material, and a second optical region made of the transparent material. In particular, the second region is immediately adjacent the first region. Preferably, a cross-section of the second optical region features a circular shape in a central position and is surrounded, in particular completely, by the first electrical region. The two regions are alternatively divided into two halves. The first electrical region is preferably designed to be at least partially made of carbon, and it is therefore black. This region can be used for optical separation between a transmission and a detection path of the regions of high optical quality, in particular for separation of LED and photodiode in PPG or fNIR sensors.

Preferably, according to an example embodiment of the present invention, the connecting element further comprises at least one electrical contacting element integrated into the connecting element. The electrical contacting element is in particular designed as a wire. The electrical contacting element is in this case designed to connect the first sensor with the scalp of the user, in particular electrically. Doing so improves the electrical contact between the first sensor and the scalp, thus improving signal detection as well.

A further object of the present invention is to provide a pair of eyeglasses comprising the sensor arrangement described above. Said object refers to data eyeglasses in particular. The sensor data acquired by means of the sensors of the sensor arrangement can in particular serve to control the data eyeglasses. Preferably, each temple of the eyeglasses supports one of the above-described sensor arrangements. The sensor arrangements on either side of the eyeglasses can be different from one another.

In this context, according to an example embodiment of the present invention, the sensor arrangement preferably further comprises a computing unit designed to receive first sensor data from the first sensor, which is in particular designed as an accelerometer, and/or a pressure sensor, and/or an EMG sensor, to compare the received first sensor data with stored first sensor data, and to activate a microphone of the eyeglasses on the basis of the comparison performed. In particular, the computing unit is designed to activate the microphone if the stored first sensor data match the received first sensor data. The stored first sensor data are in particular first sensor data generated by a special word or sentence spoken by the user of the data eyeglasses. The user can then decide for themselves which “password” they want to use in order to activate the microphone. Inadvertent activation of the microphone can be prevented as a result.

A further object of the present invention is to provide a method used for manufacturing a sensor arrangement for attachment to a head of a user. This concerns in particular the sensor arrangement described above. According to an example embodiment of the present invention, at least one first electrical sensor and/or one second optical sensor is/are in this case first connected to a support structure. The support structure serves in this case to support and/or attach the first sensor and/or second sensor to the head of the user such that the first sensor and/or second sensor is arranged on an area of the ear, in particular in an area between one ear and the head. Furthermore, at least one connecting element of an elastic material is connected to the support structure for the purpose of at least indirect connection, in particular contact, between the first sensor and/or second sensor and the scalp of the user. Preferably, the connecting element is manufactured separately as a gel pad and attached to an outer side of the temple by means of adhesive. Alternatively, the connecting element, in particular the gel pad, is integrated into the temple. In this context, the support structure and the sensor arrangement arranged thereon is preferably overmolded in an injection process.

### BRIEF DESCRIPTION OF THE DRAWINGS

FIG. 1 schematically shows a sensor arrangement mounted to a head of the user, according to an example embodiment of the present invention.

FIG. 2 schematically shows a first embodiment of a sensor arrangement for attachment to a head of a user, according to the present invention.

FIG. 3 schematically shows a second embodiment of a sensor arrangement for attachment to a head of a user, according to the present invention.

FIG. 4 schematically shows a third embodiment of a sensor arrangement for attachment to a head of a user, according to the present invention.

FIG. 5 schematically shows a fourth embodiment of a sensor arrangement for attachment to a head of a user, according to the present invention.

FIG. 6 shows a method used for manufacturing a sensor arrangement for attachment to a head of a user, according to the present invention.

FIG. 7 schematically shows one embodiment of a pair of eyeglasses, according to the present invention.

### DETAILED DESCRIPTION OF EXAMPLE EMBODIMENTS

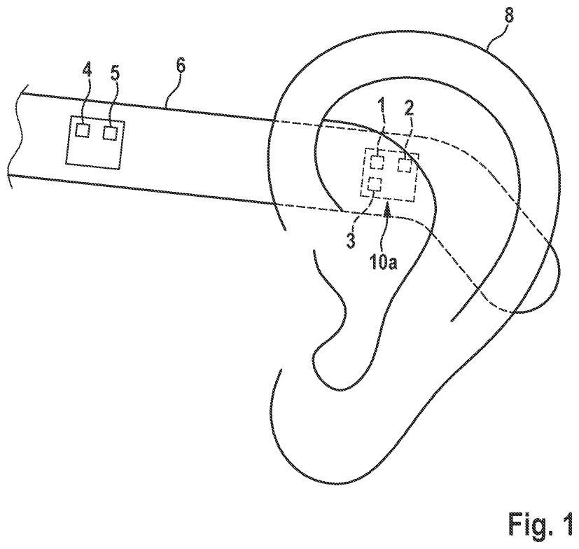

FIG. 1 schematically shows a side view of a sensor arrangement 10a, which is attached to a head of a user (not shown in this case). The sensor arrangement 10a in this case comprises two first electrical sensors 1 and 2 and a second optical sensor 3. The first electrical sensor 1 is designed as an accelerometer, and the first electrical sensor 2 as a microphone. In addition, the sensor arrangement 10a comprises a support structure 6 in the form of a temple of data eyeglasses (not further illustrated herein). The temple supports sensors 1, 2, and 3 on the head of the user, and sensors 1, 2, and 3 are arranged in an area between one ear 8 and the head of the user. This area, which is actually covered by the ear 8, is shown by way of respective dashes in FIG. 1. In addition, the sensor arrangement 10a comprises in this case two third electrical sensors 4 and 5. In this context, the temple acting as the support structure 6 supports the third sensors 4 and 5 in a region of the support structure which is arranged at a distance from the scalp of the user (not shown in this case). In this embodiment, the third sensor 4 is designed as an accelerometer, and the third sensor 5 as a microphone. All of the sensors 1, 2, 3, 4, and 5 shown in this case transmit the detected sensor data to a central control unit in particular (not shown in this case), which evaluates the sensor data. Whereas the first sensors 1 and 2, as well as the optical sensor 3, detect data from the user through skin contact, the third sensors 4 and 5 are designed to detect ambient data (e.g., ambient noise) around the user, which can then be filtered from, e.g., the sensor data from the first sensors 1 and 2, as well as the second sensor 3.

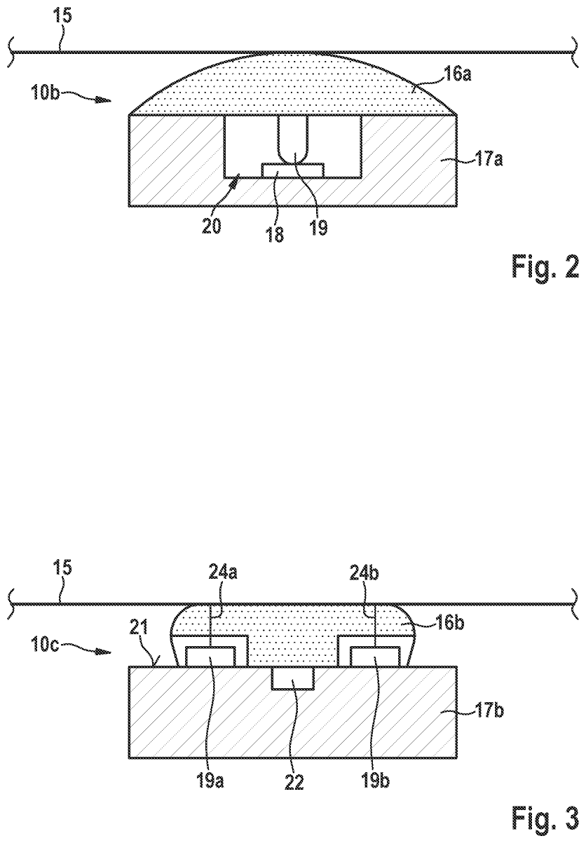

FIG. 2 is a more detailed cross-sectional view of a sensor arrangement 10b for attachment to a head of a user (not shown in this case). In this embodiment, the sensor arrangement 10b comprises a second optical sensor 18 in the form of a PPG sensor. In addition, the sensor arrangement 10b comprises a support structure 17a in the form of a temple (not further shown in this case). The sensor arrangement 10b is again arranged in an area between one ear of a user (not shown in this case) and the head of a user. For the purpose of indirect connection, in particular contact, between the second sensor 18 and the scalp 15 of the user, the sensor arrangement 10b comprises a connecting element 16a made of an elastic material in the form of a gel pad. In addition, the sensor arrangement 10b in this embodiment comprises a waveguide 19 used for guiding light waves between the connecting element 16a and the second sensor 18. The second optical sensor 18 and the associated waveguide 19 are thereby introduced into a cavity 20 of the support structure 17a, which is closed in an outward direction by the connecting element 16a. In this exemplary embodiment, the gel pad acting as a connecting element 16a is made of a transparent, in particular translucent, material.

FIG. 3 shows a cross-section of a further exemplary embodiment of a sensor arrangement 10c for attachment to a head of a user (not shown in this case). In contrast to the embodiment shown in FIG. 2, the second optical sensor 22 is in this case integrated directly into the temple 17b and does not comprise a waveguide. Furthermore, the sensor arrangement 10c comprises two first electrical sensors 19a and 19b in the form of two ECG sensors, which are connected to the outer side 21 of the data eyeglasses 17b by means of an adhesive (not shown in this case). For the purpose of connection with the scalp 15, a connecting element 16b in the form of a gel pad is also present in this case, which element encloses the two first electrical sensors 19a and 19b on the outer side 21. The gel pad also seals the second optical sensor 22 from the external environment. In order to better electrically connect the two first electrical sensors 19a and 19b with the scalp 15, an electrical contacting element 24a and 24b in the form of a wire 24a is also integrated into the connecting element 16b.

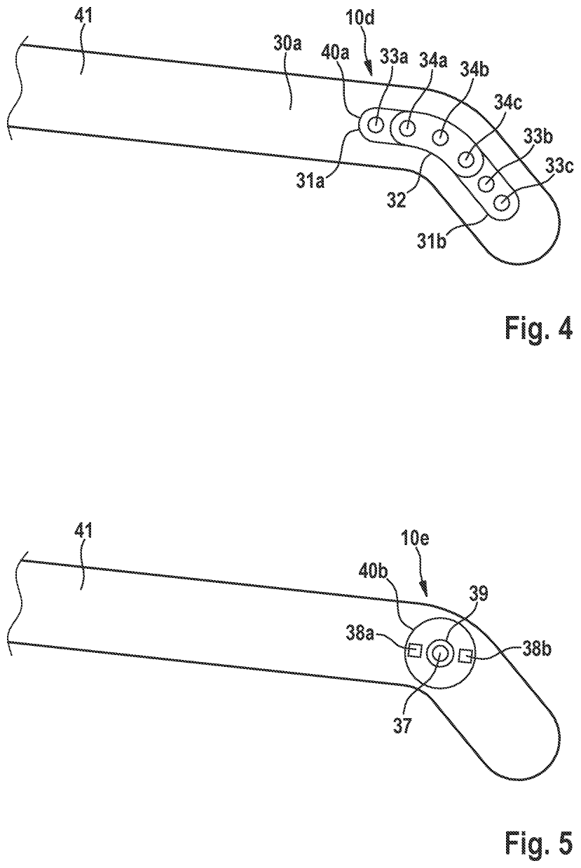

FIG. 4 shows a side view of another embodiment of a sensor arrangement 10d. In this case, the sensor arrangement 10d comprises three first electrical sensors 33a, 33b, and 33c. The sensor arrangement 10d further comprises three second optical sensors 34a, 34b, and 34c. The sensors 33a, 33b, 33c, 34a, 34b, and 34c are in turn supported by the temple as the support structure 41 on the head of the user (not shown in this case). The sensor arrangement 10d further comprises a connecting element 40a made of an elastic material for the purpose of at least indirect connection between the first 33a, 33b, 33c and second sensors 34a, 34b, and 34c and the scalp of the user (not shown). The connecting element 40a in this case comprises two first electrical regions 31a and 31b made of an electrically conductive material, as well as a second optical region 32 made of a transparent material and arranged between the two first regions 31a and 31b. The electrically conductive material in this embodiment is a carbon-admixed, two-component polysiloxane, and the transparent material is a silicone oil-filled translucent pouch.

FIG. 5 shows a side view of another embodiment of a sensor arrangement 10e. In this case, the sensor arrangement 10e comprises two first electrical sensors 38a and 38b, as well as a second optical sensor 37. The connecting element 40b comprises a second circular optical region 39 made of a transparent material, as well as a first electrical region 36 made of an electrically conductive material. The second region 39 is completely surrounded by the first electrical region 36.

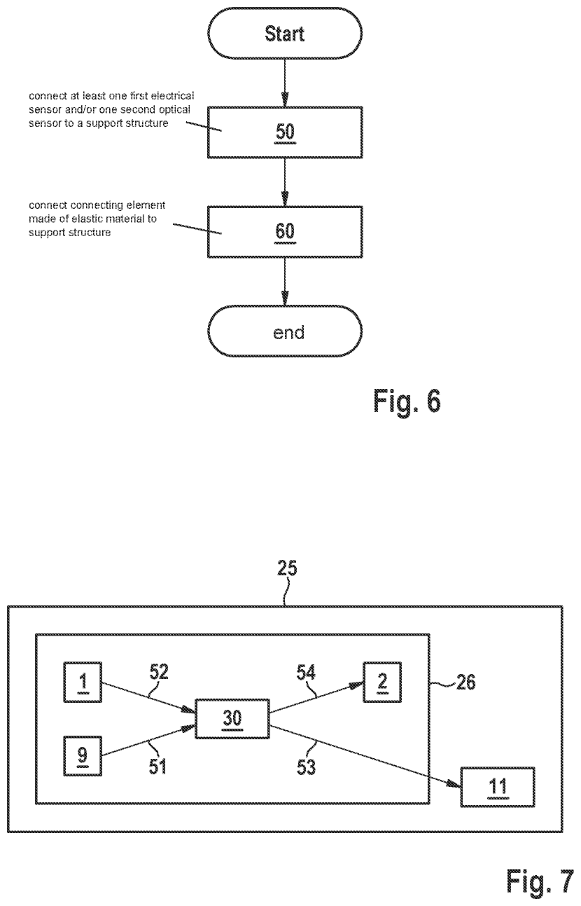

FIG. 6 shows, in the form of a flow diagram, a method used for manufacturing a sensor arrangement for attachment to a head of a user. In a first method step 50, at least one first electrical sensor and/or one second optical sensor are connected to a support structure of the sensor arrangement. The support structure is designed to support, in particular to attach, the first sensor and/or second sensor on the head of the user such that the first sensor and/or second sensor is arranged on an area of the ear, in particular in an area between one ear and the head. In a subsequent method step 60, at least one connecting element made of an elastic material is connected to the support structure for the purpose of at least indirect connection, in particular contact, between the first sensor and/or second sensor and the scalp of the user. In this case, the connecting element is manufactured separately as a gel pad and attached to an outer side of the temple by means of adhesive. Alternatively, the connecting element, in particular the gel pad, is integrated into the temple. In this context, the support structure and the sensor arrangement arranged thereon is preferably overmolded in an injection process.

FIG. 7 schematically shows a pair of eyeglasses 25 having a sensor arrangement 26. The eyeglasses 25 are in this case designed as data eyeglasses. The sensor arrangement 26 in this case further comprises a computing unit 30, which is designed to receive first sensor data 52 from the first sensor 1, which is designed as an accelerometer, and to receive first sensor data 51 from the first sensor 9, which is designed as a pressure sensor. The computing unit 30 compares the received first sensor data 51 and 52 with stored first sensor data and activates the first sensor 2, which is designed as a microphone, on the basis of the comparison. Alternatively or additionally, the computing unit 30 activates an external microphone 11 of the eyeglasses 25. In particular, the computing unit 30 is designed to activate the microphone 2 and/or 11 if the stored first sensor data matches the received first sensor data.

What is claimed is:

1. A sensor arrangement for attachment to a head of a user, comprising:
at least one first electrical sensor, and/or at least one second optical sensor;
a support structure configured to mount the first sensor and/or the second sensor on the head of the user such that the first sensor and/or the second sensor is arranged on an area between an ear and the head of the user; and
at least one connecting element made of an elastic material configured to provide at least indirect contact between the first sensor and/or second sensor and a scalp of the user.

2. The sensor arrangement according to claim 1, wherein the support structure is a first temple of data eyeglasses of the user, and the first sensor and/or the second sensor is integrated into the first temple and/or arranged on an outer side of the first temple.

3. The sensor arrangement according to claim 1, wherein the sensor arrangement includes the first sensor, and wherein the first sensor is an accelerometer, and/or a rotation rate sensor, and/or a pressure sensor, and/or an EMG sensor, and/or an ECG sensor, and/or a microphone.

4. The sensor arrangement according to claim 3, further comprising:
at least one third electrical sensor, wherein the support structure is configured to mount the third sensor on the head of the user in such a way that the third sensor is situated at a distance from the scalp of the user, and wherein the third sensor is a microphone and/or an accelerometer.

5. The sensor arrangement according to claim 1, wherein the sensor arrangement includes the second sensor, and wherein the second sensor a PPG sensor and/or an fNIR sensor.

6. The sensor arrangement according to claim 1, wherein the connecting element is a gel pad.

7. The sensor arrangement according to claim 6, wherein the sensor arrangement includes the first sensor, and the first sensor is arranged on an outer side of the support structure and is at least partially enclosed by the gel pad.

8. The sensor arrangement according to claim 1, wherein the sensor arrangement includes the second sensor, the second sensor being introduced into a cavity of the support structure, and wherein the connecting element closes the cavity in an outward direction.

9. The sensor arrangement according to claim 1, wherein the connecting element is integrated into an outer side of the support structure.

10. The sensor arrangement according to claim 1, wherein the connecting element is at least partially made of an electrically conductive material.

11. The sensor arrangement according to claim 1, wherein the connecting element is at least partially made of a translucent material.

12. The sensor arrangement according to claim 10, wherein the connecting element includes a first electrical region made of the electrically conductive material, and a second optical region, made of transparent material, which directly borders the first region.

13. The sensor arrangement according to claim 1, wherein the sensor arrangement includes the first sensor, and wherein the connecting element further includes at least one electrical contacting element, including a wire integrated into the connecting element, wherein the electrical contacting element is configured to connect electrically the first sensor with the scalp of the user.

14. Data eyeglasses, comprising:
a sensor arrangement for attachment to a head of a user, including:
at least one first electrical sensor, and/or at least one second optical sensor,
a support structure configured to mount the first sensor and/or the second sensor on the head of the user such that the first sensor and/or the second sensor is arranged on an area between an ear and the head of the user, and
at least one connecting element made of an elastic material configured to provide at least indirect contact between the first sensor and/or second sensor and a scalp of the user.

15. The data eyeglasses according to claim 14, wherein the sensor arrangement includes the first sensor, and wherein the sensor arrangement further includes a computing unit configured to receive first sensor data of the first sensor, the first sensor being an accelerometer, and/or a pressure sensor, and/or an EMG sensor, and where the computing using is configured to compare the received first sensor data with stored first sensor data, and to activate a microphone of the eyeglasses based on the comparison.

16. A method used for manufacturing a sensor arrangement for attachment to a head of a user, the method comprising the following steps:
connecting at least one first electrical sensor and/or one second optical sensor with a support structure of the sensor arrangement, wherein the support structure is configured to attach the first sensor and/or second sensor on the head of the user such that the first sensor and/or second sensor is arranged in an between an ear and the head of the user; and
connecting at least one connecting element made of an elastic material to the support structure, the connecting element configured to provide at least indirect contact between the first sensor and/or second sensor and a scalp of the user.
